(19) United States
(12) Patent Application Publication
(10) Pub. No.: US 2024/0170106 A1
(43) Pub. Date: May 23, 2024

(54) METHOD FOR DETERMINING THE ACIDITY DISTRIBUTION CURVE OF OILS FROM THE MOLECULAR COMPOSITION OF THE CRUDE OIL

(71) Applicants: PETRÓLEO BRASILEIRO S.A. – PETROBRAS, Rio de Janeiro (BR); UNIVERSIDADE FEDERAL DE GOIÁS - UFG, Goiânia (BR)

(72) Inventors: IRIS MEDEIROS JUNIOR, Rio de Janeiro (BR); GABRIEL FRANCO DOS SANTOS, Goiânia (BR); ALEXANDRE DE OLIVEIRA GOMES, Rio de Janeiro (BR); BONIEK GONTIJO VAZ, Goiânia (BR); GESIANE DA SILVA LIMA, Goiânia (BR); DEBORAH VICTÓRIA ALVES DE AGUIAR, Goiânia (BR); JUSSARA VALENTE ROQUE, Goiânia (BR)

(21) Appl. No.: 18/517,625

(22) Filed: Nov. 22, 2023

(30) Foreign Application Priority Data
Nov. 22, 2022 (BR) BR1020220237484

(51) Int. Cl. G16C 20/30; G01N 33/28; G16C 20/70

(52) U.S. Cl. CPC G16C 20/30; G01N 33/2876; G16C 20/70

(57) ABSTRACT

The present disclosure refers to the use of very high resolution mass spectrometry analysis methodology in combination with the use of multivariate calibration models to predict Total Acidity Number (TAN). The models are built from data of total abundance value with the application of machine learning methods for regression.

### FIELD OF THE DISCLOSURE

The present disclosure refers to a method for determining the acidity distribution curve of oils from the molecular composition of the crude oil using multivariate calibration models to predict the Total Acidity Number (TAN) from ESI (−) FT-ICR MS data of the crude oil, being a quick and efficient solution compared to the traditional approach.

The present disclosure has application in the field of supply and biofuels, coke and separation processes, distribution, logistics and transport as well as maintenance and supply facilities.

### BACKGROUND

Oil refining consists of the series of improvements that the crude oil undergoes to obtain its derivates. These improvements include physical and chemical separation steps that originate the oil fractions. Each fraction of the oil is further a mixture of hydrocarbons formed by a smaller number of substances, which are separated due to their different boiling points. Refining oil is, therefore, separating the desired fractions such as gasoline, kerosene, diesel oil, naphtha, cooking gas, among many others.

Conversion processes also take place in refineries where some heavy fractions are converted by chemical reactions into products with high added value. The conversion process includes different types of reactions, such as thermal and catalytic cracking, hydrotreating, isomerization reactions, alkylation and rearrangements. Cracking is a chemical process that transforms heavier oil fractions into lighter ones, using high temperatures and pressures (thermal cracking) or in the presence of a metallic catalyst (catalytic cracking). Isomerization and rearrangement reactions transform normal-chain hydrocarbons into branched-chain isomer hydrocarbons (with higher octane) through the use of catalysts and/or heating. Hydrotreating reactions use hydrogen to remove contaminants such as sulfur and nitrogen [Reference 1].

Each process is a complex combination of operations that depend on the multivariable and highly interactive nature of each oil to be processed. Therefore, in refineries, the use of models that simulate in real time the situations of the refining system based on the composition of the starting oil is essential. When the crude oil feed changes composition or when the demand and prices of refinery products fluctuate, a controller based on predictive models must calculate targets that compensate for processing the oil at that time. Therefore, real-time optimization based on predictive models can be used to characterize the composition of the crude oil and model the separation and conversion processes to calculate targets and optimize unit profits.

The chemical characterization of oil is still a major challenge. Crude oil is believed to have more chemical compounds than the number of genes found in the human genome [Reference 2]. The composition of the oil can be defined as a continuous distribution of molecular weight, structure and functionality from low boiling point fractions to non-distillable residues [Reference 3].

Therefore, the acidity of oil samples has been of great interest to the industry due to the association of this property with aspects that directly influence the valuation and production of oil. The presence of acids in oil reduces its economic value, in addition to generating risks and costs. They directly impact productivity, as they can promote the formation and/or stabilization of emulsions, deposition of naphthenates and corrosion in distillation systems [Reference 4]. Currently, information corresponding to the acidity of crude oils and their derivates is accessed through traditional methods, which are extremely laborious and expensive, involving high volumes of samples and time, and are often unfeasible in the oil industry, as the necessary amount of oil to carry out the experiments is not available.

The measurement of the oil acidity is carried out by the Total Acidity Number (TAN), being one of the most important parameters in oil processing. TAN is defined as the amount of KOH (mg) required to neutralize the acidic components in titrating 1.0 g of crude oil (D664-09) [Reference 5]. Initially, the crude oil is subjected to a fractional distillation step to obtain its derivates using the true boiling point curve (TBP curve). The determination of the TBP curve can be carried out according to the ASTM D 2892 standard [Reference 6], requiring the use of up to 30 liters of crude oil. After obtaining the derivates, the total acidity number (TAN) of the crude oils and their derivates is determined. At this step, all samples are subjected to a potentiometric titration [References 5 and 7] according to the ASTM D664 standard [Reference 8]. Given this, the use of new approaches that optimize the method for determining the acidity of oils and their respective distillation cuts is necessary.

In this sense, it should be emphasized that the development of modern analytical techniques such as very high resolution mass spectrometry, which encompasses cyclotron resonance of ions with Fourier transform (FT-ICR MS), has made it possible to characterize hundreds of thousands of polar compounds extremely quickly and efficiently [Reference 9]. This field of science is known as petroleomics, and its objective is to correlate and predict the behavior, reactivity and properties of oils and its derivates based on detailed data on their chemical composition [Reference 10]. Therefore, the use of mass spectrometry and petroleomics is a comprehensive characterization technique, that is, its results can be used to support both exploration and production, refining and distribution activities [References 11 and 12]. Petroleomics has contributed greatly to expanding knowledge of the composition of oil and its fractions. However, despite the great advances experienced in the comprehensive characterization of oil and its derivates by mass spectrometry of very high resolution and accuracy, new analytical methods, systematized and standardized, to access compositional and structural information at the molecular level are necessary in petroleomics approaches, where the compositional information can predict the behavior and properties of oil and derivates. In this context, there is a need of applying multivariate data processing approaches, such as machine learning methods, with the aim of creating prediction models and prospecting molecular properties of oil and derivates. Therefore, the combination of FT-ICR MS and multivariate methods emerges as a potential technology for classifying oil and derivate properties in terms of TAN value.

The state of the art consists of various approaches related to the use of ESI (−) FT-ICR MS to build models for classifying oil in association with the TAN parameter, such as, for example, the dissertation titled “Aplicações Espectrometria de Massas de Ressonância Ciclotrônica de Íons por Transformada de Fourier (FT-ICR MS) em Petroleômica” (“Applications of Spectrometry of Ion Cyclotron Resonance Masses by Fourier Transform (FT-ICR MS) in Petroleomics”), authored by Thieres Magaive Costa Pereira, which aimed at evaluating the thermodegradation of naphthenic acids, in addition to the use of ESI, APCI, APPI, LDI and MALDI ionization sources to acquire new data relating to the characterization of asphaltene samples. However, this work does not develop any method for predicting TAN, nor does it use any chemometric tool to do so.

The dissertation titled “Novas abordagens na caracterização de petróleos por espectrometria de massas FT-MS” (“New approaches in the characterization of oils by FT-MS mass spectrometry”), authored by Denys Ribeiro de Oliveira Costa, focuses on the use of ionization sources and FT-ICR MS for the characterization of the chemical profile of samples of oil, naphthenic acids and asphaltenic fractions. However, the work at no point addresses to methodologies for identifying or predicting the TAN number, nor does it address to chemometric methods and the development of predictive models for this property.

The dissertation titled “Caracterização de Compostos Polares no Petróleo por Espectrometria de Massas de Altíssima Resolução e Exatidão—ESI (±)-FT ICR-MS” (“Characterization of Polar Compounds in Oil by Very High Resolution and Accuracy Mass Spectrometry—ESI (±)-FT ICR-MS”), authored by Guilherme Pires Dalmaschio, focuses on the characterization of polar compounds present in oil and in its cuts by mass spectrometry, using the electrospray ionization technique, in positive mode, coupled with an FT-ICR analyzer. In the document, the author addresses to the characterization of the compounds present in the sample and draws a superficial correlation between the polar compounds and total acidity number (TAN). However, the author does not show any method to predict or assign the TAN values to the oil samples. Only the TAN values, obtained by the conventional method established by ASTM D664-09, were used to discuss the results of the mass analyses. Due to the presence of acidic compounds in certain oil samples, this is a correlation commonly described in the literature. However, in this research, there are no TAN predictive model approaches for oil, as in the presented disclosure.

The paper titled “Petroleomics: The Next Grand Challenge for Chemical Analysis”, authored by Alan G. Marshall and Ryan P. Rodgers, addresses to the challenges faced in petroleomics, mainly in terms of quantification of polar compounds by FT-ICR MS and advances in the field of modeling and machine learning. This document is a collection of advances in this area until 2004. However, there are no approaches or methods for obtaining or predicting the TAN values, nor do the authors use any multivariate calibration approach to access this property like the models presented in the present disclosure.

In the dissertation titled “Predição de curva de destilação de gasolina de pirólise a partir de resultados de cromatografia em fase gasosa e calibração multivariada” (“Prediction of pyrolysis gasoline distillation curve from gas chromatography results and multivariate calibration”), authored by Milla Beatrice Engelmann de Oliveira Garcia, a multivariate calibration was applied to predict values of distillation of heavy PYGAS (heavy pyrolysis gasoline) from gas chromatography results. The acquisition of data used in the work was carried out through GC-MS analyses, different from that used in the present disclosure (ESI-FT-ICR MS). The data obtained in both analyses have a different nature, evaluating different groups of chemical compounds. The central idea of the work described by Garcia is to develop a prediction model to determine the distillation values of heavy PYGAS. The present disclosure seeks to develop models to predict the acidity of oils (TAN prediction) without the need for prior distillation. Accordingly, both works deal with different themes, addressing to physicochemical properties without correlation between the same.

In the paper titled “Estudo da Acidez Naftênica e Potencial Corrosivo de Petróleos Brasileiros por ESI (−) FT-ICR MS” (“Study of Naphthenic Acidity and Corrosive Potential of Brazilian Oils by ESI (−) FT-ICR MS”), authored by Martins and collaborators, the naphthenic acidity and corrosive potential of 35 samples of Brazilian crude oils were evaluated using the relative abundance of the O₂ class obtained by ion cyclotron resonance mass spectrometry with Fourier transform (FT-ICR MS). This work characterizes the acidic compounds present in the oils, an approach commonly performed from the interpretation of mass data employs ESI (−). However, in this work, no method was proposed for predicting the acidity of oil (TAN number) from the mass data obtained.

In the paper titled “Petroleômica: Fundamentos e Aplicações na Caracterização Geoquímica de Petróleos e Rochas Geradoras” (“Petroleomics: Fundamentals and Applications in the Geochemical Characterization of Oil and Source Rocks”), authored by Ygor dos Santos Rocha; Rosana Cardoso Lopes Pereira; João Graciano Mendonça Filho, a literature review was presented regarding the state of the art in the application of ultra-high resolution mass spectrometry in petroleomics. This article presents an overview of the most relevant works in the literature regarding the basic principles of the mass ionization technique and its sources of ionization, processing and data handling, as well as petroleomics applied to oil geochemistry. In this literature review, there is no development of predictive methods for oil properties. On the other hand, in the present disclosure, multivariate models were developed to predict the TAN of oils and their respective distillation cuts based on ultra-high resolution mass spectrometry data from crude oil samples.

In the paper titled “Prediction of Total Acid Number in Distillation Cuts of Crude Oil by ESI (−) FT ICR MS Coupled with Chemometric Tools”, authored by Luciana A. Terra and collaborators, a method was developed for predicting the TAN of oils and their distillation cuts from ESI (−) FR-ICR MS spectra together with chemometric methods. This work presents predictive models on mass spectra obtained from crude oil distillation cuts, which requires a distillation step. The innovation of the present disclosure lies precisely in that only with ultra-high resolution mass spectrometry data from the crude oil samples is it possible to predict the TAN of the respective distillation cuts, without the need for the distillation step, which requires high quantities of crude oil, and this factor could be a major bottleneck for the oil industry.

In the paper titled “Predictive Petroleomics: Measurement of the Total Acid Number by Electrospray Fourier Transform Mass Spectrometry and Chemometric Analysis”, authored by Boniek G. Vaz and collaborators, a method was developed for predicting the TAN of oils from the spectra of ESI (−) FR-ICR MS together with chemometric methods. This work presents TAN predictive models only for crude oils. The innovation of the present disclosure lies in the development of prediction models that use ultra-high resolution mass spectrometry data from crude oil samples that can predict this property for the oil itself and for its respective distillation cuts, without the need for the step distillation process to obtain the cuts.

In the European patent of disclosure EP 2 651 541 B1, titled “Generation of model-of-composition of petroleum by high resolution mass spectrometry and associated analysis”, a method was presented for determining the characterization of the composition of oil (hydrocarbons), specifically using samples of vacuum residue, VGO (vacuum gas oil) or crude oil. The main idea of the disclosure was to create a model for characterizing and quantifying the chemical composition of heavy oil using mass spectrometry data. However, in the present disclosure, multivariate models were developed to predict the TAN acidity of oils and their respective distillation cuts from ultra-high resolution mass spectrometry data of crude oil samples, without the need for the distillation step to obtain the cuts.

Therefore, the state of the art does not provide for the possibility of predicting the TAN of the respective distillation cuts, without the need for the distillation step, such as the present disclosure. This is possible through multivariate calibration models for predicting the Total Acid Number (TAN) from ESI (−) FT-ICR MS data of crude oil, as claimed in the present application. A method for predicting this property, where it is not necessary to distill the crude oil, is extremely important for the oil industry, considering that this step is an extremely laborious process, requires a large amount of sample and requires a lot of time for execution. Therefore, the new approach of the present disclosure optimizes the process of determining the acidity of oils and their respective distillation cuts.

### SUMMARY OF THE DISCLOSURE

The present disclosure refers to the use of multivariate calibration models to predict the TAN (Total Acidity Number) from the total abundance values obtained by ESI (−) FT-ICR MS of the crude oil. To build the calibration models, partial least squares (PLS) regression was used in combination with the ordered predictor selection (OPS) method.

### BRIEF DESCRIPTION OF DRAWINGS

The present disclosure will be described below, with reference to the attached figures which, in a schematic way and not limiting the inventive scope, represent examples of its embodiment.

FIG. 1 shows a Flowchart of the building process of the TAN prediction models of the present disclosure.

FIG. 2 illustrates graphs referring to error values relative to a set of samples, being (A) and (F) oils; (B) and (G) JET-A1; (C) and (H) diesel; (D) and (I) gas oil; (E) and (J) vacuum residue.

### DETAILED DESCRIPTION

The present disclosure refers to the use of multivariate calibration models to predict TAN (Total Acidity Number) from data obtained by ESI (−) FT-ICR MS of crude oil. This solution is quick and efficient compared to the traditional approach, as it is capable of predicting the oil TAN values and their respective cuts without the need for the traditional approach of crude oil fractionation, which would require a large amount of sample, in addition to not being necessary to carry out the potentiometric titration.

The present disclosure makes it possible, through ESI (−) FT-ICR MS chemical analysis, to build robust models for classifying oils according to the TAN value, thus aiming at better understanding the composition of the oil and, consequently, its better application industrial. Furthermore, the disclosure is characterized by being a practical and direct method, which is carried out by analyzing crude oil without the need for laboratory experiments such as chromatographic elutions and potentiometric titration to determine the TAN. The proposed method provides a drastic reduction in cost and time, considering that it excludes the distillation and titration steps. Furthermore, it is carried out using a much smaller amount of sample (around 10 mg of crude oil) than that used in the conventional process.

The methodology used in the present disclosure uses ESI (−) FT-ICR MS data to build the models, which were extracted from the composition table generated in the Composer software and imported into the Matlab 2020a software (MathWorks, Natick, USA). A data matrix containing the total abundance values was built and called X matrix, which are the independent variables. The rows of the X matrix correspond to nine oil samples and the columns correspond to the molecular formulas, which are the variables. The data containing said total abundance values obtained by ESI (−) FT-ICR MS were used to build the multivariate calibration models. To build the calibration models, partial least squares (PLS) regression was used in combination with the ordered predictor selection (OPS) method.

Chemical analysis by petroleomics of the crude oil with application of multivariate calibration models PLS with data from ESI (−) FT-ICR MS with variable selection by OPS can be easily applied to crude oil samples with similar characteristics to those used in the modeling step to predict the TAN of the oil itself and its respective cuts quickly and efficiently.

The methodology further uses two sets of different variables to predict the TAN: set 1 (O, O₂, O₃ and O₄) and set 2 (O, O₂, O₃, O₄, N, N₂, N₂O, N₂O₂, NO, NO₂, NS, NOS, OS, O₂S and O₃S). Therefore, two X matrices were used to build the models. In total, 10 different models were built, 5 coming from each set of variables. Thus, the same X matrix was used to build PLS models to predict the TAN values of crude oil, JET-A1, diesel, gas oil and vacuum residue.

In this way, the methodology allows obtaining the oil acidity curve with milligrams of sample, allowing the process engineer to evaluate the impacts of the corrosive effect on oil and derivates processing equipment, avoiding or enabling interventions to reduce the damage that may be caused by premature corrosion in equipment.

### Example of Embodiment/Tests/Results

There follow below the specific experiments and tests that were carried out to predict the TAN property of the cuts from oils.

Prediction of the TAN Property of the Cuts from Oils Sample Preparation for Analyses

In the present disclosure, nine oil samples were used (S1, S2, S3, S4, S5, S6, S7, S8, S9)). The samples were prepared by solubilizing 1.0 mg of each sample in 1.0 mL of toluene. For ESI (−) analyses, 500 μL of the stock solution was transferred to a vial containing 500 μL of methanol. The final concentration of the analyzed solution was 500 μg mL⁻¹ in toluene/methanol (50:50, v/v). 1.0 μL of a sodium trifluoroacetate (NaTFA) solution at 0.001 mg mL⁻¹ was added to each sample. The NaTFA solution was used as an internal standard.

### Mass Spectrometry Analysis

Mass spectrometry analyzes were carried out using an FT-ICR MS 7T SolariX 2×R equipment (Bruker Daltonics—Bremen, Germany) coupled to the ESI source. The equipment was calibrated daily with a solution of 0.1 μL mL⁻¹ of NaTFA calibrant, for both ionization modes, in the m/z range of 150 to 2000. The average calibration error varied between 0.02 and 0.05 ppm in the quadratic regression mode. The samples were injected using a syringe pump with a flow rate of 120 μL h⁻¹. 8MW data sets were acquired through magnitude mode with the detection range of m/z 150 to 2000 for the oils.

For each sample, 200 scans were acquired to obtain spectra with excellent signal/noise values. The general conditions for analysis by ESI (−), as well as the parameters used in acquiring the spectra for the analyzes are shown in Table 1.

TABLE 1
Parameters used for the acquisition of ESI
(−) FT-ICR MS spectra of the analyzed cuts.

| Sample Parameters | ESI (−) |
|---|---|
| Concentration (mg · mL⁻¹) | 0.25-0.50 |
| % of dopant | 2.5% |
| Source Parameters | ESI (−) |
| Flow (μL · h⁻¹) | 120 |
| Capillary voltage (kV) | 3.0-3.8 |
| End Plate Offset (V) | −800 |
| Source Gas Nebulizer (bar - | 1.0 |
| x 100 kPa) | |
| Ion source gas | 200 |
| temperature (° C.) | |
| Capillary Exit (V) | −220 |
| Deflector Plate (V) | −200 |
| Funnel 1 | −150 |
| Skimmer (V) | (−)15 − (−)50 |
| Funnel RF Amplitude (Vpp) | 150 |
| Collision voltage (V) | 1.5 |
| Ion Accumulation | 0.005-0.02 |
| Time (sec) | |
| Octopole | ESI (−) |
| Frequency (MHz) | 5 |
| RF Amplitude (Vpp) | 350 |
| Quadrupole | ESI (−) |
| Q1 Mass (m/z) | 200 |
| Collision Cell | |
| Collision Voltage (V) | 1-10 |
| DC Extract Bias (V) | (−0.5) − (−1.2) |
| RF Frequency (MHz) | 2 |
| Collision RF Amplitude | 1000 |
| (Vpp) | |
| Transfer Optics | ESI (−) |
| Time of Flight (msec) | 0.400-0.850 |
| Frequency (MHz) | 6 |
| RF Amplitude (Vpp) | 350 |
| Flow Gas Control (%) | 25 |
| Analyzer Para Cell | ESI (−) |
| Transfer Exit Lens (V) | 20 |
| Analyzer Entrance (V) | 10 |
| Side Kick (V) | −1.5 |
| Side Kick Offset (V) | 0.0 |
| Front Trap Plate (V) | −1,500 |
| Back Trap Plate (V) | −1500 |
| Back Trap Plate Quench (V) | −30 |
| Sweep Excitation Power (%) | 28 |
| Shimmming DC Bias | ESI (−) |
| 0° (V) | −1.290 |
| 90° (V) | −1.490 |
| 180° (V) | −1.710 |
| 270° (V) | −1.510 |
| Gated Injection DC Bias | ESI (−) |
| 0° (V) | −3.000 |
| 90° (V) | −0.800 |
| 180° (V) | −1.900 |
| 270° (V) | −2.200 |

Therefore, the data containing the total abundance values obtained by ESI (−) FT-ICR MS from a set of nine oil samples were used to build the multivariate calibration models for predicting the TAN in oils and their cuts of distillation. To build the calibration models, partial least squares (PLS) regression [Reference 13] was used in combination with the ordered predictor selection (OPS) method [Reference 14].

### Building of the Multivariate Calibration Models (TAN Prediction)

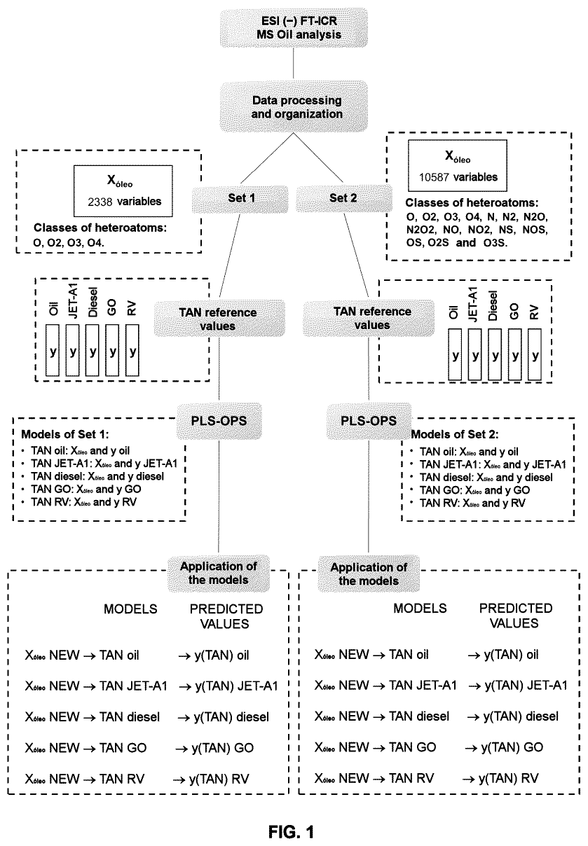

The flowchart presented in FIG. 1 outlines the process from obtaining data in the ESI (−) FT-ICR MS analyses to applying the models obtained to predict the TAN.

Initially, as explained above, the ESI (−) FT-ICR MS data used to build the models were extracted from the composition table generated in the Composer software and imported into the Matlab 2020a software (MathWorks, Natick, USA). A data matrix containing the total abundance values of the oil samples was built and called the X_{óleo} matrix, which are the independent variables. The rows of the X_{óleo} matrix correspond to nine oil samples and the columns correspond to the variables.

Given that the acidity in oil samples depends on the different types of functional groups present in the oil, two sets of different variables were used to predict TAN, such as:
In set 1, the total abundances attributed to detected compounds belonging to classes O, O₂, O₃ and O₄ were selected, totaling 2338 variables; and
In set 2, in addition to the classes of heteroatoms mentioned above, compounds belonging to the classes 0, O₂, O₃, O₄, N, N₂, N₂O, N₂O₂, NO, NO₂, NS, NOS, OS, O₂S and O₃S were also considered, totaling 10587 molecular formulas.

These compounds were considered for creating the models due to the influence they exert on the total acidity distribution curve of the samples.

A vector containing the respective TAN values was built and named y, which is the dependent variable. The y vector has a number of rows equal to the number of samples in the X_{óleo} matrix. In fact, 2 X_{óleo} matrices were built, referring to each set of variables, and 5 y vectors with the TAN reference values, referring to oil, JET-A1, diesel, gas oil (GO) and vacuum residue (RV), as can be seen in the flowchart in FIG. 1.

Table 2 presents the reference TAN values for the oils and their respective cuts in addition to the predicted values for the oils and their respective distillation cuts.

TABLE 2
Calculated parameters for PLS-OPS models

| | | | | | |
|---|---|---|---|---|---|
| NVL | Nvars | RMSEC | Rc | RMSECV | Rcv |

Set 1

| | | | | | | |
|---|---|---|---|---|---|---|
| Oil | 8 | 325 | 0.1637 | 0.9690 | 0.2259 | 0.9460 |
| JET-A1 | 6 | 70 | 0.0510 | 0.9904 | 0.0834 | 0.9648 |
| Diesel | 5 | 55 | 0.0216 | 0.9996 | 0.0870 | 0.9414 |
| Gas Oil | 7 | 10 | 0.2191 | 0.9546 | 0.2394 | 0.9214 |
| Residue | 5 | 370 | 0.0752 | 0.9803 | 0.1394 | 0.9538 |

Set 2

| | | | | | | |
|---|---|---|---|---|---|---|
| Oil | 2 | 129 | 0.1077 | 0.9906 | 0.1245 | 0.9873 |
| JET-A1 | 4 | 163 | 0.0456 | 0.9938 | 0.1010 | 0.9618 |
| Diesel | 3 | 333 | 0.0459 | 0.9982 | 0.0909 | 0.9933 |
| Gas Oil | 2 | 70 | 0.0745 | 0.9952 | 0.0798 | 0.9965 |
| Residue | 3 | 216 | 0.0293 | 0.9971 | 0.1748 | 0.9936 |

NVL: number of latent variables;
Nvars: number of selected variables;
RMSEC: square root of the mean square error of the calibration;
Rc: calibration correlation coefficient;
RMSECV: square root of the mean squared error of cross-validation;
Rcv: cross-validation correlation coefficient.

Table 3 presents the reference TAN values for the oils and their respective cuts in addition to the predicted values for the oils and their respective distillation cuts.

TABLE 3
Reference total acidity number (TAN) predicted by
the PLS-OPS models of the nine oils and their respective
cuts analyzed by ESI(−) FT-ICR MS

| | oil | | | JET-A1 | | | Diesel | | | Gas Oil | | | Vacuum Residue | | |
|---|---|---|---|---|---|---|---|---|---|---|---|---|---|---|---|
| Set | REF | PRED1 | PRED2 | REF | PRED1 | PRED2 | REF | PRED1 | PRED2 | REF | PRED1 | PRED2 | REF | PRED1 | PRED2 |
| S1 | 0.130 | 0.128 | 0.112 | 0.079 | 0.075 | 0.101 | 0.060 | 0.061 | 0.044 | 0.080 | 0.079 | 0.006 | | 0.084 | 0.084 |
| S2 | 0.260 | 0.259 | 0.339 | 0.215 | 0.223 | 0.223 | 0.270 | 0.263 | 0.298 | 0.230 | 0.226 | 0.179 | | 0.024 | 0.028 |
| S3 | 0.140 | 0.138 | 0.007 | | 0.215 | 0.215 | | 0.347 | 0.347 | 0.20 | 0.200 | 0.168 | 0.100 | 0.100 | 0.100 |
| S4 | 0.260 | 0.259 | 0.027 | 1.250 | 1.333 | 1.301 | 1.180 | 1.270 | 1.154 | 0.520 | 0.522 | 0.521 | | 0.107 | 0.111 |
| S5 | 0.220 | 0.218 | 0.075 | 0.175 | 0.171 | 0.201 | 0.220 | 0.216 | 0.264 | 0.180 | 0.177 | 0.123 | | 0.067 | 0.072 |
| S6 | 2.320 | 2.595 | 2.353 | 0.380 | 0.393 | 0.286 | 2.290 | 2.444 | 2.329 | 2.530 | 2.741 | 2.484 | 1.120 | 1.145 | 1.143 |
| S7 | 0.110 | 0.108 | 0.149 | 0.040 | 0.036 | 0.024 | 0.050 | 0.048 | 0.0750 | 0.040 | 0.039 | 0.164 | 0.090 | 0.090 | 0.133 |
| S8 | 0.660 | 0.689 | 0.705 | 0.120 | 0.112 | 0.105 | | 0.359 | 0.359 | 0.880 | 0.939 | 0.773 | 0.380 | 0.385 | 0.343 |
| S9 | 0.430 | 0.437 | 0.417 | 0.215 | 0.234 | 0.162 | 0.430 | 0.422 | 0.335 | 0.480 | 0.477 | 0.573 | 0.260 | 0.256 | 0.236 |

REF: TAN reference value;
PRED1: TAN value predicted by the model obtained with variables of set 1;
PRED2: TAN value predicted by the model obtained with the variables of set 2.

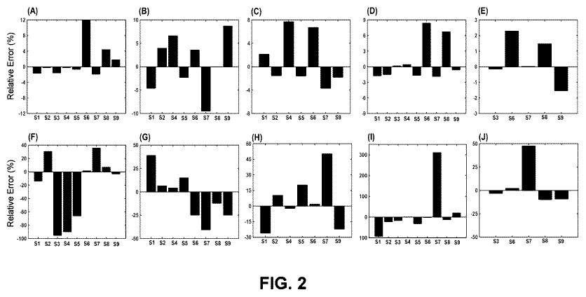

All prediction models of set 1 showed relative errors of less than 11% (FIG. 2 (Graphs A-E)). For set 2, relative errors greater than 50% were obtained (FIG. 2 (Graphs F-J)), with the highest values being found for the oil and gas oil samples (error equal to 100 and 300%, respectively).

From the data obtained, it was possible to demonstrate that the disclosure is capable of predicting oil TAN values and their respective cuts without the need of fractionating the crude oil, which requires a large amount of sample, in addition to not being necessary to perform a potentiometric titration. With the method of this disclosure, it is possible to predict the TAN of the oil and its respective cuts with the acquisition of the ESI (−) FT-ICR MS of the oil, facilitating the understanding of the oil system with the quick prediction of important characteristics for the value chain. Therefore, the disclosure solves problems related to the insufficient amount of the sample required in the traditional method and, therefore, the acidity of any sample can be determined with the process developed in this disclosure.

### REFERENCES

[1] R. E. Young, Process control 2, PPI Pulp Pap. Int. 45 (2006) 39.
[2] E. Niyonsaba, K. E. Wehde, R. Yerabolu, G. Kilaz, H.

I. Kenttämaa, Determination of the chemical compositions of heavy, medium, and light crude oils by using the Distillation, Precipitation, Fractionation Mass Spectrometry (DPF MS) method, Fuel. 255 (2019) 115852. https://doi.org/10.1016/j.fuel.2019.115852.
[3] M. M. Boduszynski, Composition of Heavy Petroleums. 2. Molecular Characterization, Energy and Fuels. 2 (1988) 597-613. https://doi.org/10.1021/ef00011a001.
[4] R. C. Pereira, R. C. Petroleômica: Caracterização de Petróleos Nacionais por Espectrometria de Massas de Altíssima Resolução: O Que os Compostos Ácidos Podem Revelar sobre o Petróleo. 2012, 185.
[5] P. A. P. Decote, L. Negris, A. P. Vidoto, L. A. N. Mendes, E. M. M. Flores, M. A. Vicente, M. F. P. Santos, Determination of the total acid number of Brazilian crude oil samples: Theoretical and experimental evaluation of three standard methods. Fuel, 2022, 313 122642. https://doi.org/10.1016/j.fuel.2021.122642.
[6] ASTM D2892: Standard Test Method for Distillation of Crude Petroleum. DOI: 10.1520/D2892-20.
[7] H. A. Fernandes, L. N. Zanelato, P. A. P. Decote, H. N. Santos, C. M. Senger, F. C. Dias, E. I. Muller, E. M. M. Flores, L. A. N. Mendes, M. A. Vicente, M. F. P. Santos, Effects of calcium, magnesium, and strontium chlorides in determining the total acid number using potentiometric titration. Fuel, 2022, 311, 122522. https://doi.org/10.1016/j.fuel.2021.122522
[8] ASTM D664: Standard Test Method for Acid Number of Petroleum Products by Potentiometric Titration. DOI: 10.1520/D0664-18E02.
[9] R. M. Alberici, R. C. Simas, G. B. Sanvido, W. Romão, P. M. Lalli, M. Benassi, I. B. S. Cunha, M. N. Eberlin, Ambient mass spectrometry: bringing MS into the “real world” Anal. Bioanal. Chem., 2010, 398 265-294. https://doi.org/10.1007/s00216-010-3808-3.
[10] A. G. Marshall, R. P. Rodgers, Petroleomics: The next grand challenge for chemical analysis. Acc. Chem. Res., 2004, 37, 53-59. https://doi.org/10.1021/ar020177t.
[11] C. A. Hughey, C. L. Hendrickson, R. P. Rodgers, A. G. Marshall, Elemental composition analysis of processed and unprocessed diesel fuel by electrospray ionization Fourier transform ion cyclotron resonance mass spectrometry. Energy Fuels, 2001, 15, 1186-1193. https://doi.org/10.1021/ef010028b.
[12] G. P. Dalmaschio, M. M. Malacarne, V. M. D. L. De Almeida, T. M. C. Pereira, A. O. Gomes, E. V. R. De Castro, S. J. Greco, B. G. Vaz, W. Romão, Characterization of polar compounds in a true boiling point distillation system using electrospray ionization FT-ICR mass spectrometry. Fuel, 2014, 115, 190-202. https://doi.org/10.1016/j.fuel.2013.07.008.
[13] P. Geladi, B. C. Kowalski, Partial least-squares regression: a tutorial. Anal Chim Acta, 1986, 185, 1-17. J. V. Roque, W. Cardoso, L. A. Peternelli, R. F. Teófilo. Comprehensive new approaches for variable selection using ordered predictors selection. Anal Chim Acta, 2019, 1075:57-70. https://doi.org/10.1016/j.aca.2019.05.039.

1. A method for determining the acidity distribution curve of oils from the molecular composition of crude oil, the method comprising:
preparing crude oil samples;
obtaining total abundance values by very high resolution mass spectrometry from crude oil samples;
building multivariate calibration models; and
comparing total acidity number (TAN) reference values with the values of the multivariate calibration models for TAN prediction.

2. The method according to claim 1, wherein the total abundance values obtained are selected from two sets of different variables: Set 1, wherein the total abundances attributed to the detected compounds belong to classes O, O₂, O₃ and O₄, totaling 2338 variables; and Set 2, wherein, in addition to the variables mentioned above, there also are considered heteroatoms belonging to the classes O, O₂, O₃, O₄, N, N₂, N₂O, N₂O₂, NO, NO₂, NS, NOS, OS, O₂S and O₃S, totaling 10587 variables.

3. The method according to claim 1, wherein the built multivariate calibration models comprise partial least squares (PLS) regression in combination with ordered predictor selection method.

4. The method according to claim 3, wherein application of the PLS multivariate calibration models with data from ESI (−) FT-ICR MS of the crude oil with selection of variables by OPS generates a prediction of the TAN of the oil itself and its respective cuts.

5. The method according to claim 1, wherein data from the total abundance values are used to build the multivariate calibration models and are extracted from a composition table generated in first software and imported into second software.
